(19) United States
(12) Patent Application Publication
(10) Pub. No.: US 2010/0112105 A1
(43) Pub. Date: May 6, 2010

(54) Antimicrobial efficacy of aframomum Melegueta extract against propionibacterium acnes

(75) Inventors: Anne L. Hoyt, Lowell, MI (US); Jatinder Rana, Grand Rapids, MI (US); Kausar Malik, Grand Rapids, MI (US); Donald J. Pusateri, Hemet, CA (US)

Correspondence Address: ALTICOR INC., 7575 FULTON STREET EAST MAILCODE 78-2M, ADA, MI, 49355, US

(73) Assignee: ACCESS BUSINESS GROUP INTERNATIONAL LLC, Ada, MI (US)

(21) Appl. No.: 12/261,328

(22) Filed: Oct. 30, 2008

(51) Int. Cl. A61K 36/906; A61K 31/12; A61P 17/10

(52) U.S. Cl. 424/756; 514/678

(57) ABSTRACT

The use of compositions containing one or more phytochemicals such as gingerols, paradols, and mixtures thereof for aiding in the control, reduction or elimination of Propionibacterium acnes. Aframomum melegueta extracts containing the one or more phytochemicals can be used against P. acnes to effect the growth of the bacterium on a surface.

### CROSS-REFERENCE TO RELATED APPLICATIONS

This application claims priority to U.S. Provisional Application No. 61/001,035, filed Oct. 30, 2007, which is incorporated herein by reference in its entirety.

### BACKGROUND OF THE INVENTION

(1) Field of the Invention

The present invention relates to the use of extracts of Aframomum melegueta against Propionibacterium acnes. The extracts are useful in personal care or skin care products to aid in the control, reduction, or elimination of P. acnes in individuals showing signs of skin acne.

(2) Description of Related Art

Propionibacterium acnes (P. acnes) is a species of relatively slow growing aerotolerant gram-positive anaerobic bacilli that is associated with acne. In the skin of individuals with acne, the overgrowth of P. acnes in blocked pores leads to the rupture of the pores to form lesions. Therefore, antibiotics have often been used to control this bacterial growth. Some antibiotics that are currently used by dermatologists to control acne include tetracycline, doxycycline, minocycline, erythromycin, clindamycin, vancomycin and sulfonamides. In addition, keratolytic agents, such as benzoyl peroxide and retinoids (e.g. tretinoin, adapalene, tazarotene) are often used to clear the skin of patients with acne. Severe cases of inflammatory acne can be treated with the retinoid isotretinoin. However, isotretinoin has the drawback of being a teratogen, causing severe birth defects. In some cases antiandrogen therapy is even used to control androgen excess in women, since androgen production stimulates and causes enlargement of sebaceous glands.

Various skin care products are presently sold on the market to control acne. However, none of these acne products use extracts of Aframomum melegueta for the control of the acne producing bacteria P. acnes. Aframomum melegueta, also known as “grains of paradise”, “melegueta pepper”, “Guinea grains”, “Guinea pepper” and “alligator pepper” is a species of the ginger family (Zingiberaceae) native to the west coast of Africa. Aframomum melegueta has been noted as a means to treat and prevent the inflammatory response. U.S. Patent Application Publication No. 2005/0260290 to Raskin teaches anti-inflammatory extracts of a plant material of Aframomum melegueta. Also, gingerols, found in plants of the ginger family, have been incorporated into skin care products with the purpose of smoothing wrinkles. However, Aframomum melegueta has not been previously used for the control of the acne producing bacteria P. acnes.

Some antimicrobial activity has been seen associated with the related plants, Aframomum danielli (Fasoyiro et al., “Phytochemical Characterization and the Antimicrobial Property of Aframomum danielli Extract”, African Journal of Agricultural Research, 2(3), 076-079 (2007)), and Aframomum longifolius (Tatsimo et al. “Antimicrobial principle from Aframomum longifolius”, Planta Medica, 72(2), 132-135 (2006)). Fasoyiro et al., teach inhibition of Bacillus subtilis, Bacillus cereus, Staphylococcus aureus, Escherichia coli, and Pseudomonas aeruginosa with A. danielli petroleum ether extracts. U.S. Patent Application Publication No. 2005/0058729 to Staggs teaches a method of treating bacterial infection of S. aureus with a black pepper lotion. Although the term “pepper” is commonly used for both black pepper (Piper nigrum of the family Piperaceae) and chili pepper (genus Capsicum), these plants are not related to the “melegueta pepper” (Aframomum melegueta) of the ginger family. Therefore, extracts from these plants contain different phytochemicals than extracts obtained from Aframomum melegueta.

Certain phytochemicals from Aframomum melegueta have been found to have activity against mycobacteria. Mycobacteria are nonmotile, aerobic bacteria of the genus Actinobacteria causing diseases such as leprosy and tuberculosis. Galal, “Antimicrobial Activity of 6-Paradol and Related Compounds”, Pharmaceutical Biology, vol. 34(1), pp. 64-69 (1996), teaches 6-paradol and 6-shogaol as the agents of Aframomum melegueta active against Mycobacterium chelonei, M. intracellulare, M. smegmatis, and M. xenopi. However, Galal teaches that gingerone was found to be inactive.

The teachings of Ogbulie et al. illustrate that aqueous extracts of Aframomum melegueta have no antibacterial effect on the isolates of S. pyogenes, E. coli, S. typhi P. aeruginosa, and Vibrio sp. Furthermore, Ogbulie et al. teach that hot, cold and soxhlet ethanol extracts of Aframomum melegueta have no antibacterial effect on the isolates of S. aureus, E. coli, S. typhi, and Vibrio sp. Only the cold ethanol extracts of Aframomum melegueta slightly inhibited P. aeruginosa, while hot ethanol and soxhlet ethanol extractions did not inhibit P. aeruginosa. According to Ogbulie et al., the results indicate that A. melegueta has no antibacterial effect on the isolates showing that it does not contain any active principle against these organisms.

None of the related art references teach the use of Aframomum melegueta extracts against Propionibacterium acnes. Thus there exists a need for such an aid in the control, reduction, or elimination of P. acnes from a surface.

### BRIEF SUMMARY OF THE INVENTION

The present invention provides a method of effecting the growth of Propionibacterium acnes on a surface comprising: providing an organic extract of Aframomum melegueta; providing a surface to be treated; and administering the extract of Aframomum melegueta to the surface to be treated, so as to effect the growth of the Propionibacterium acnes. In further embodiments, the extract comprises one or more paradols. In still further embodiments, the extract comprises one or more gingerols. In some embodiments, the Aframomum melegueta is extracted with a carboxylate or an alcohol to provide the extract. In some embodiments, the Aframomum melegueta is extracted with ethyl acetate or methanol.

The present invention provides a method of effecting the growth of Propionibacterium acnes on a surface to be treated comprising: providing a composition comprising one or more paradols, one or more gingerols, or a mixture thereof; providing a surface to be treated; and administering the composition to the surface to be treated, so as to effect the growth of the Propionibacterium acnes. In further embodiments, the composition comprises one or more paradols. In further embodiments, the composition comprises one or more gingerols.

### BRIEF DESCRIPTION OF THE DRAWINGS

FIG. 1 shows a disk diffusion assay of an ethyl acetate extract (bottom half of plate) and a methanol “MeOH” extract (upper half of plate) of Aframomum melegueta at ten (10), twenty (20), or thirty (30) microliters per disc. Botanical: Aframomum melegueta. Organism: Propionibacterium acnes (ATCC No. 6923). Media: Mueller Hinton agar with 5% sheep blood.

FIG. 2 shows thin-layer chromatography (TLC) of an ethyl acetate extract (Lane 1-3) and a methanol extract (Lanes 4-6) of Aframomum melegueta. Bands for paradols, gingerols, and shogaols were identified.

FIG. 3 illustrates a disk diffusion bioautography assay indicating paradols (1) and gingerols (2) as active marker phytochemicals. No activity was seen with shogaols (3).

### DETAILED DESCRIPTION OF THE INVENTION

All patents, patent applications, and literature references cited in this specification are hereby incorporated herein by reference in their entirety. In case of conflict, the present description, including definitions, will control.

The term “effecting” as used herein refers to the act of controlling, reducing, and/or eliminating microorganisms, such as bacteria. For example, the term can refer to bacteriostatic effect to control the growth of bacteria. The term also can refer to a bactericidal effect to reduce and/or eliminate the bacteria.

The term “administering” as used herein refers to any route of administration of an active ingredient such as an extract or one or more active phytochemicals. The term includes, but is not limited to, topical application to a surface.

The term “acne” as used herein refers to the skin condition acne vulgaris.

The term “alcohol” as used herein refers to short chain alkyl alcohols, such as methanol and ethanol.

The term “organic extract” as used herein refers to the material extracted from a source by means of one or more organic compounds.

The term “gingerols” as used herein refers to one or more gingerols, including but not limited to [6]-gingerol.

The term “paradols” as used herein refers to one or more paradols, including but not limited to [6]-paradol.

The term “shogaols” as used herein refers to one or more shogaols.

The present invention provides a method of controlling, reducing or eliminating Propionibacterium acnes on a surface to be treated. The methods can utilize one or more organic extracts of Aframomum melegueta, applied to a surface such as skin, so as to control the growth, reduce or eliminate the Propionibacterium acnes. Alternatively, one or more phytochemicals, such as gingerols and/or paradols, can be used. The one or more phytochemicals can be administered to a surface so as to control, reduce, and/or eliminate Propionibacterium acnes.

### EXAMPLE 1

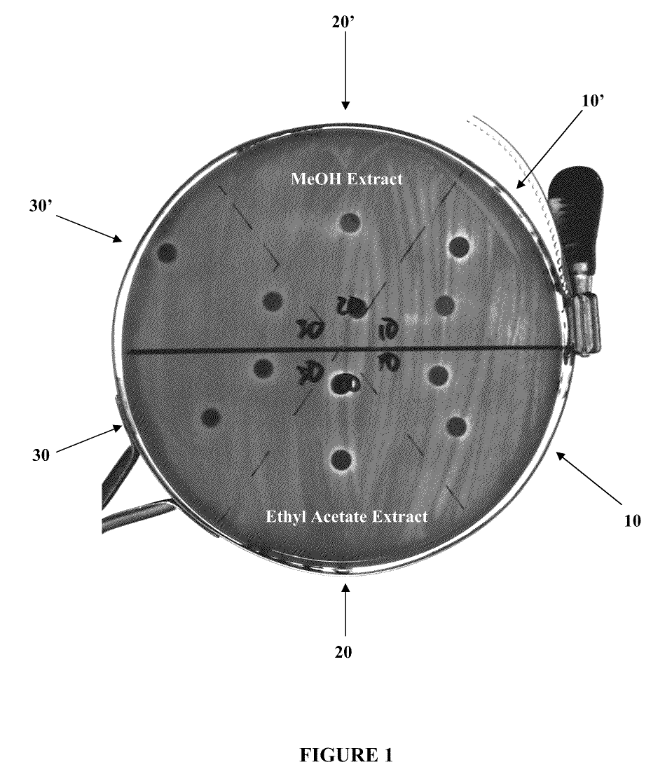

This example illustrates the effectiveness of an organic extract of Aframomum melegueta seeds against P. acnes. The procedure used to evaluate the presence of antimicrobial activity was performed in accordance with NAACLS Clinical Laboratory Standards for antimicrobial susceptibility testing. Standard chemical extraction methods were applied using organic extracts such as methanol or ethyl acetate. The extract was then applied to sterile blank antibiotic discs and placed on a Mueller Hinton with 5% sheep blood agar plate that had been inoculated with P. acnes. The plate was then incubated in anaerobic conditions for seventy-two hours at 37° C.±1° C., at which time the plate was evaluated for the presence or absence of a clear zone surrounding the disc as seen in FIG. 1. FIG. 1 shows a disk diffusion assay at ten (10, 10′), twenty (20, 20′), or thirty (30, 30′) microliters per disc of an ethyl acetate extract (10, 20, 30 on bottom half of plate) and a methanol “MeOH” extract (10′, 20′, 30′ on upper half of plate) of Aframomum melegueta. Botanical: Aframomum melegueta. Organism: Propionibacterium acnes (ATCC No. 6923). Media: Mueller Hinton agar with 5% sheep blood. Both methanol extracts and ethyl acetate extracts showed antimicrobial activity against P. acnes.

### EXAMPLE 2

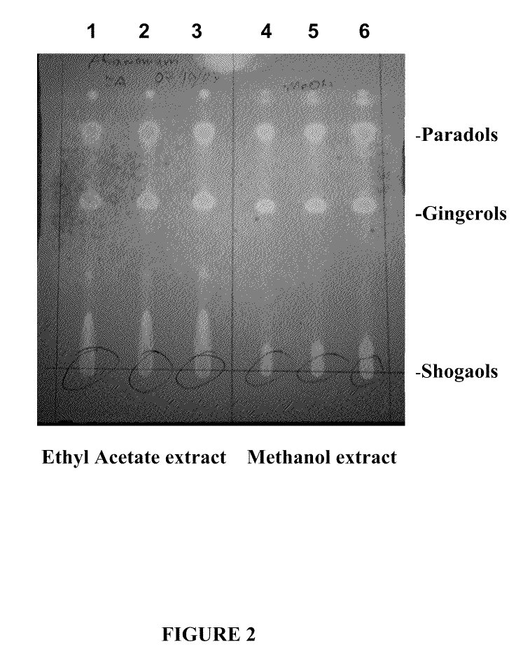

This example illustrates the photochemical profile of Aframomum melegueta. Thin layer chromatography was performed with the extracts of Example 1 to show the photochemical profile of Aframomum melegueta. The resulting TLC plate is illustrated in FIG. 2. FIG. 2 shows thin-layer chromatography (TLC) of an ethyl acetate extract (Lanes 1-3) and a methanol extract (Lanes 4-6) of Aframomum melegueta. Paradols, gingerols and shogaols were resolved as identified on the right.

### EXAMPLE 3

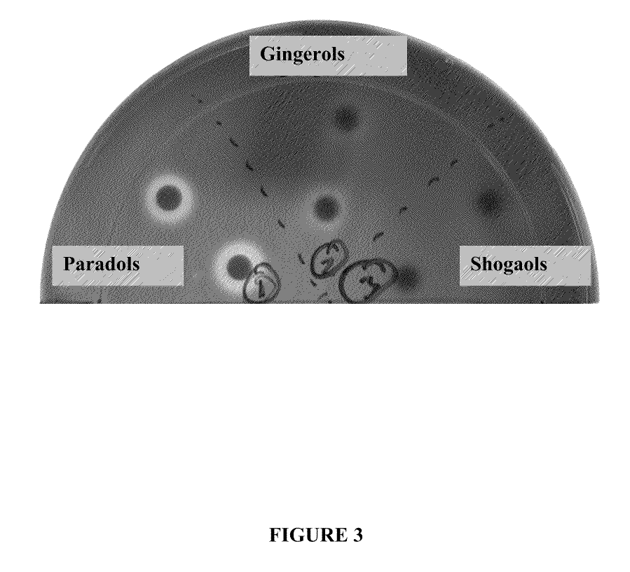

This example illustrates the effectiveness of phytochemicals extracted from Aframomum melegueta against P. acnes. FIG. 3 illustrates a disk diffusion bioautography assay indicating paradols and gingerols as active marker phytochemicals. The results indicate that paradols and gingerols, but not shogaols, are the active phytochemicals extracted from Aframomum melegueta against the bacterium P. acnes.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1-5. (canceled)

6. A method of effecting the growth of Propionibacterium acnes on a surface to be treated comprising:
(a) providing a composition consisting essentially of one or more paradols;
(b) providing Propionibacterium acnes on a surface to be treated; and
(c) administering the composition consisting essentially of one or more paradols to the surface to be treated, so as to effect the growth of the Propionibacterium acnes.

7-8. (canceled)

9. The method of claim 6, wherein the one or more paradols are provided by extraction from Aframomum melegueta.

10. The method of claim 9, wherein the one or more paradols have been extracted using a carboxylate.

11. The method of claim 10, wherein the carboxylate is ethyl acetate.
